(19) United States
(12) Patent Application Publication
(10) Pub. No.: US 2021/0186791 A1
(43) Pub. Date: Jun. 24, 2021

(54) DUAL-PURPOSE TREATMENT TABLE

(71) Applicant: Cardon Rehabilitation & Medical Equipment Ltd., Burlington (CA)

(72) Inventor: Mardig SEVADJIAN, Oakville (CA)

(21) Appl. No.: 17/109,750

(22) Filed: Dec. 2, 2020

Related U.S. Application Data
(60) Provisional application No. 62/952,703, filed on Dec. 23, 2019.

(51) Int. Cl. A61G 13/12; A61G 13/00; A61B 5/00

(52) U.S. Cl. CPC A61G 13/1285; A61G 13/121; A61B 5/704; A61G 13/125; A61G 13/009; A61G 13/1235

(57) ABSTRACT

The present invention relates to treatment tables, and more specifically to a treatment table that is adaptable for the dual purposes of conducting therapy or physical examinations, including pelvic examinations and treatment.

### CROSS-REFERENCE TO RELATED APPLICATION

This application claims the benefit of the filing date U.S. provisional patent application 62/952,703, filed Dec. 23, 2019, the entire disclosure of which is incorporated herein.

### FIELD OF THE INVENTION

The present invention relates to treatment tables, and more specifically to a treatment table that is adaptable for the dual purposes of conducting therapy or physical examinations, including pelvic examinations and treatment.

### BACKGROUND OF THE INVENTION

Treatment tables are used to provide a variety of therapies and to enable physical examinations of different parts of the body. As described in U.S. Pat. No. 4,236,261, patient stretchers and hospital beds have head, middle, and foot sections and in this embodiment the head section may be tilted. As described in U.S. Pat. No. 9,278,041, there are examination tables wherein the upper support structure has a sliding access panel disposed on a side of the upper support structure such that an examination passageway is provided when the sliding access panel is repositioned.

It is desirable to have a table that is easily adjustable to be a therapy table or an examination table including for pelvic examinations.

### SUMMARY OF THE INVENTION

In an embodiment of this invention there is a treatment table for a person to rest on having a first end with a removable head support section and with two foot/arm supports extending from first end of the treatment table wherein each foot/arm support is sized to support a forearm or foot.

The foot/arm supports may be adjustable outwardly and inwardly as well as upwardly and downwardly relative to the table.

The treatment table may additionally comprise a removable leg rest on each side of the table wherein each leg rest is independently postionable along the length of the treatment table.

In an embodiment of this invention there is a treatment table comprising a leg rest on each side of the table that is independently postionable along the length of the treatment table. These leg rests may be removable.

In an embodiment of this invention there is a treatment table with a head rest comprised of a first head rest section surrounded by a second head rest section wherein the first head rest section is removable and the second head rest section is on a spring loaded hinge bendable into an over 90 degrees downward angle from the original position. The first head rest section may be replaceable with a tray, or the first head rest section covers a tray which is accessible when the first head rest section is removed.

In an embodiment of this invention there is a treatment table comprising at least a first section and a second section wherein the first section and the second section are both independently adjustable to tilt towards each other. The second section may be larger than the first section and the second section can tilt up to 90 degrees and the first section can tilt up to 20 degrees. This treatment table may additionally comprise a head section. This treatment table may additionally comprise removable leg rests which slide independently along each side of the table. This treatment table may additionally comprise two foot/arm supports extending from the treatment table wherein each foot/arm support is sized to support a forearm or foot. This treatment table's first section may have concave contours on each side.

In an embodiment of this invention there is a treatment table comprising a first section at a first end and a second section at a second end, and two foot/arm supports extending from the first end of the treatment table, wherein the first section is adjustable to tilt upwards at the first end. The second section may be additionally adjustable to tilt upwards at a second end. This treatment table may additionally comprise a removable leg rest on each side of the table and wherein each leg rest is independently postionable along the length of the treatment table. The foot/arm supports may be adjustable outwardly and inwardly as well as upwardly and downwardly relative to the table. This treatment table may additionally comprise a removable head section.

### BRIEF DESCRIPTION OF THE FIGURES

These and other aspects of the present invention will be apparent from the brief description of the drawings and the following detailed description in which:

FIG. 1 is a first end perspective view of an embodiment of a treatment table of the present invention in a first configuration.

FIG. 2 is a first end perspective view of an embodiment of a treatment table of the present invention in a second configuration.

FIG. 3 is a right-side view of an embodiment of a treatment table of the present invention in a third configuration.

FIG. 4 is the treatment table shown in FIG. 2 indicating direction for possible position adjustments.

FIG. 5 is a first end perspective view of an embodiment of a treatment table of the present invention in a fourth configuration.

FIG. 6 is a right-side view of an embodiment of a treatment table of the present invention in a fifth configuration.

FIG. 7 is a first-end view of an embodiment of a treatment table of the present invention in a sixth configuration.

FIG. 8 is first end perspective close-up view under a raised middle pad of a treatment table of the present invention.

### DETAILED DESCRIPTION OF THE INVENTION

The present invention is a treatment table that has the dual purpose of allowing therapy or physical examinations, including pelvic examinations and treatment. The treatment table of the present invention provides a therapist with accessibility to the individual being treated, provides support and positions conducive for relaxation of the muscles of the individual being treated, and provides various options for adjusting the treatment table. A therapist refers generally to any person providing any type of adjustments, therapy or treatment to a person ranging from relaxation to medical purposes.

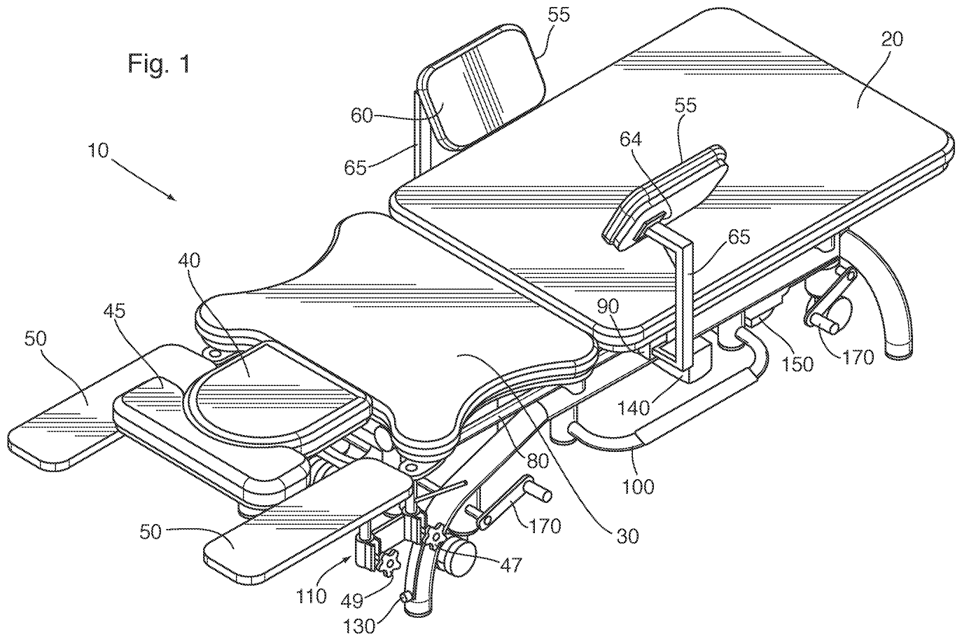

FIG. 1 shows an embodiment of a treatment table 10 of the present invention having a main pad 20, a middle pad 30, and at a first end there is a first head section 40 and a second head section 45 for a person to lie on. In this configuration, the table is typically used with a person's head at the first end on the left. When a person rests their head on the first end of the table 10, the back of their head may rest on the first head section 40 and second head section 45. For a patient lying on their front with their head at the first end of the table, their arms can rest on the foot/arm supports 50, and the leg rests 58 are either not used or can be removed. First head section 40 can be replaced with a first head section having an opening for a face when a person is lying on the table on their front. When the leg rests 58 are removed or moved towards the second end of the table, the therapist can be close to the patient particularly given the optional concave contours in the middle pad 30.

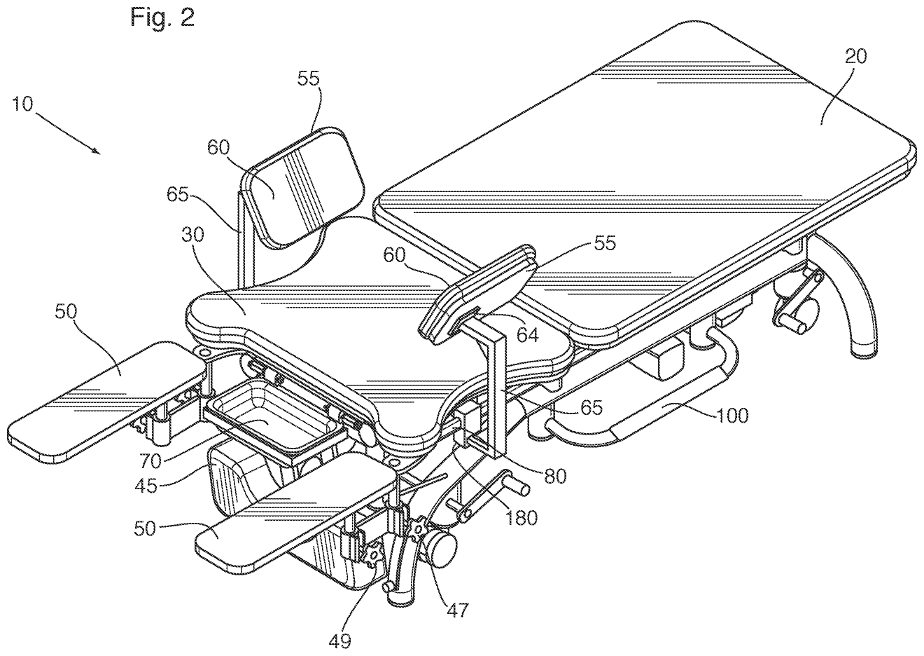

FIG. 2 shows the table 10 without the first head section 40, which is pulled out to remove it, and may be replaced by inserting a tray 70 in place of first head section 40. The first head section 40 and tray 70 may be inserted by prongs which fit into bushings on the table 10. The second head section 45 is pushed down out of the way. Preferably the second head section is pushed down and slightly back away from the first end (for example on a hinge that bends more than 90 degrees) so that a therapist can put their knees under the table. The first head section 40 may be replaceable with a tray 70 or in another embodiment a tray 70 may be accessible when a first head section 40 is removed.

In the configuration shown in FIG. 2, the table is typically used with a person lying down on their back with their head at the second end on the main pad 20, with their thighs supported by leg rests 55 on leg rest pads 60, and their feet supported on foot/arm supports 50. The leg rest pads 60 may be attached to a leg rest post 65 via a spring-loaded plate 64 to provide flexibility and be more comfortable for a person when their legs are resting on the leg rest pads 60. The spring-loaded plate 64 allows the leg rest pads to tilt as the person on the table allows the weight of their legs to relax and fully rest on the leg rest pads 60, for example, up to 60 degrees.

In the configuration shown in FIG. 2, the treatment table 10 may comprise a middle pad 30 or first section and a main pad 20 or second section for the resting surface portion of the bed, and no head section or head sections.

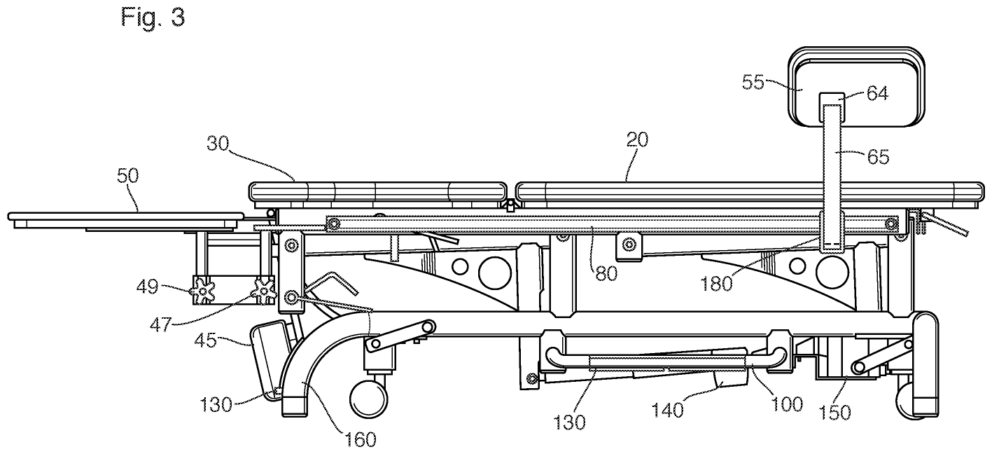

FIG. 3 shows the leg rests 55 in another position on the table in an embodiment with a leg rest rail 80 along which the leg rests 55 slide via a leg rest slide 90. This allows a practitioner to adjust the position of the leg rests for the comfort of the person on the table and also for the therapist to get close to the person on the table. The leg rests may be adjusted along each side to rest at the same or different positions relative to one another.

For comfort of the practitioner, there may be footrests 100 on each side of the table. Also, there may be easy to reach switches 130 on each side of the table 10, to turn on an electric activator 140 to raise the table higher or lower, as powered by a power supply 150. The switches 130 are shown here under the footrest 100 and on a front table leg 160, and one switch may be for raising and the other for lowering the table.

The therapist can be close to the person on the table as a result of the concave contours in the middle pad 30, particularly if the leg rests 55 are slid towards the second end to that the therapist can be along the concave contours.

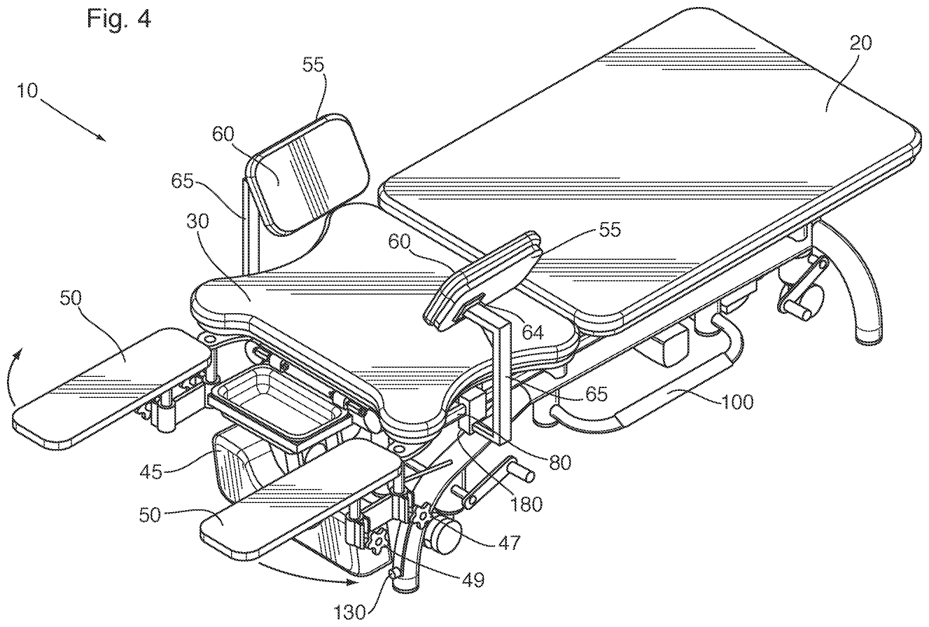

FIG. 4 shows that in an embodiment of the invention the foot/arm supports 50 are adjustable in position and height since they are on swivel arms 110, and by loosening and tightening swivel knob 47 the foot/arm supports 50 may swivel out and in, and by loosening and tightening height knobs 49 the foot/arm supports may be raised and lowered. This is for the comfort of the patient and to aid the therapist in accessing the person on the bed.

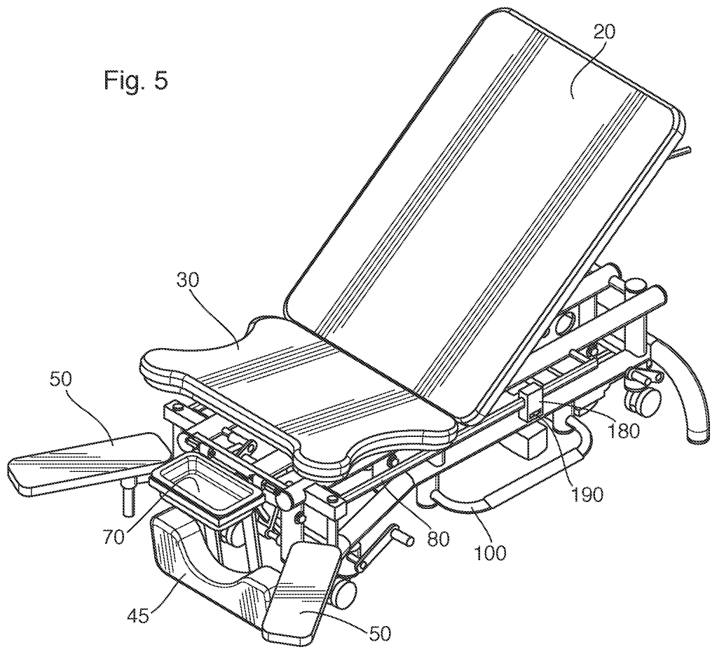

FIG. 5 shows the table with the main pad raised for a person to sit or recline on the table. For example, the main pad 20 could be raised up to 90 degrees. The angle of the main pad 20 may be may be raised and lowered in any manner that is currently available, for example, using gas spring cylinders, or in any manner available in the future. FIG. 5 shows the foot/arm supports 50 swiveled outwardly. In this configuration shown in FIG. 5, a practitioner may get close to the pelvis of a patient for a pelvic examination. The tray 70 may be used for probes and utensils for treatment or may be removed by pulling it out.

FIG. 5 also shows the middle pad 30 tilted, for example, the middle pad could be raised 15 or 20 degrees or less. Tilting the middle pad 30 may provide comfort for the person on the table and better effectiveness for the therapist. Tilting the middle pad 30 can prevent a person on the table from sliding forward and enable the person to fully relax and feel secure on the table and the therapist to be more effective since the person is relaxed and not tensed. Tilting the middle pad 30 up allows the pelvis to rest in a more neutral and supported position. When a person is in a comfortable position on the table the pelvic muscles can remain relaxed. For people with pain in the tailbone/sacrum area it may be difficult and painful to endure therapy when lying or sitting given the prolonged pressure on the tailbone/sacrum for the duration of treatment. The table 10 of the present invention provides tilting of the middle pad 30 to reduce the pressure exerted on the tailbone/sacrum and provide relief and/or less pain during therapy sessions.

The table 10 of the present invention also provides effective treatment ability and more comfort for the therapist. Tilting the middle pad 30 upwards allows for better mechanics of the therapist's treating shoulder, elbow and wrist, as the therapist can rest their elbow on the middle pad 30 and get close. The concave contours of the middle pad 30 also allows the therapist to get closer. The upward tilting of the middle pad 30 also provides better access to the ano-rectal opening of a person, and with a person more relaxed, this allows for easier and more comfortable rectal exams for both the person on the bed and the therapist.

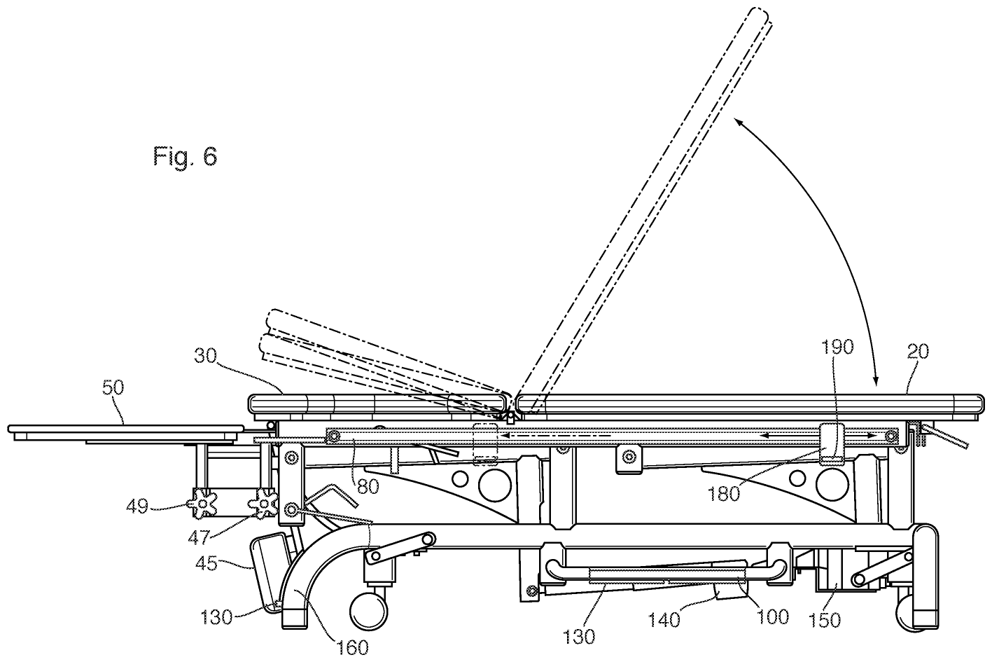

FIG. 6 shows that the table 10 of the present invention provides for adjusting the main pad 20 and the middle pad 30 relative to one another for the comfort of the person on the bed and the effectiveness and comfort of the therapist.

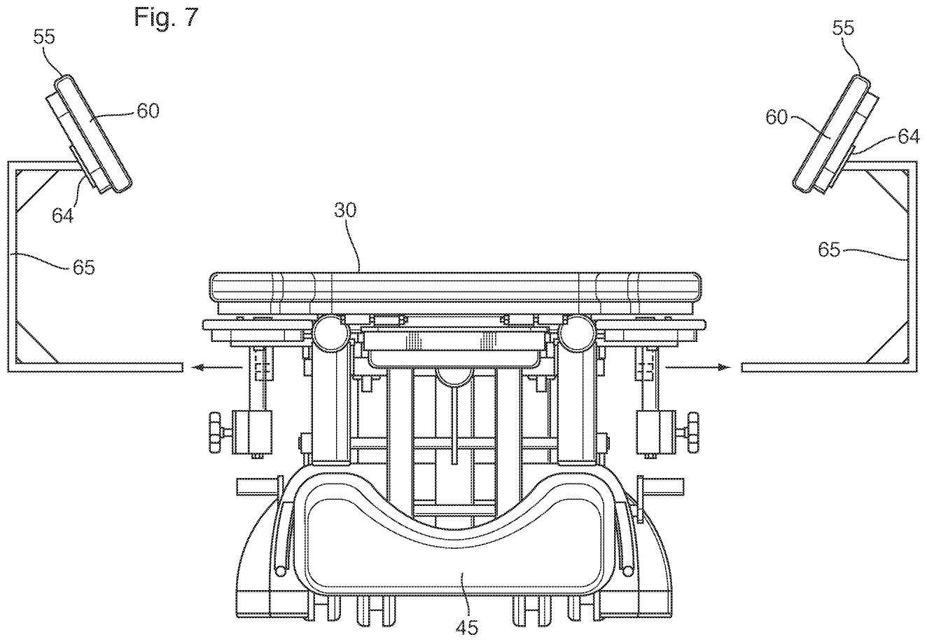

FIG. 7 shows the leg rests 55 being removed. If a person is getting onto the table 10, it can be helpful to remove a leg rest 55 to allow them to slide onto the bed, or to slide the leg rest towards the second end of the table 10 to allow the person to use the leg rest as a support to hoist themselves onto the table, after which the leg rest may be slid back into position for the patient to use for support for their thighs when lying or reclining on the table.

FIG. 7 shows the location of the insertion of a leg rest 55 via leg rest bar 65 fitting into bracket slot 190 (see FIGS. 5 and 6) in the leg rest bracket 180 on the table 10. Since the leg rests 55 on each side of the table are independently attached to the table 10, the table may be set up with both leg rests, or only one leg rest on either side, or no leg rests.

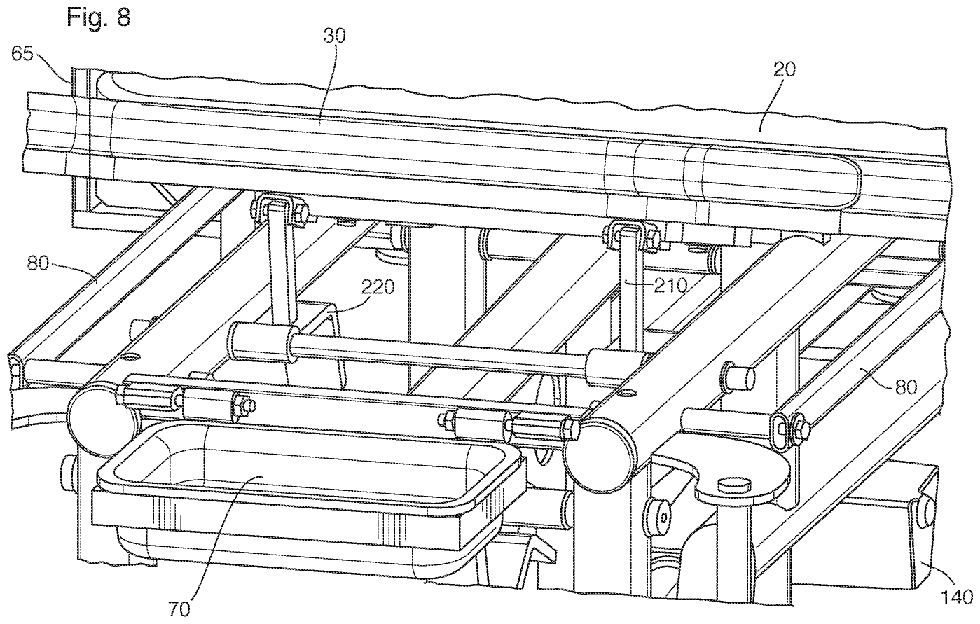

The middle pad 30 of the table 10 may be tilted similarly to the main pad 20 or with a different mechanism. In an embodiment of the invention as shown in FIG. 8, the tilting of middle pad 30 is possible by lifting the middle pad 30 up and at the desired height gently lowering the middle pad into place. The locking bars 210 have spaced notches so that they lock mechanically onto the flappers 220. When the middle pad needs to be lowered, it is either yanked up and pushed down or the flappers are pulled back to release the locking bars 210.

In an embodiment of the invention as shown in FIG. 1, the leg rests 55 are adjustable along the table from a first end to a second end by sliding along a leg rest rail 80 via a leg rest slide 90. The leg rest is inserted (through slot 190 visible in FIGS. 5 and 6) into the leg rest slide 90. When the person on the bed rests a thigh on a leg rest 55, the leg rest remains in place due to the friction created by the weight of the leg.

The table 10, may also optionally be capable of raising and lowering in height for ease of a person getting on the table and also for a therapist to conduct their treatment effectively and comfortably, and in an embodiment shown in FIG. 1 there are cranks 170 at each of four supporting legs on wheels which raise and lower through turning the cranks 170.

The table 10 of the present invention provides for adjusting the main pad 20 and the middle pad 30 relative to one another, as well as adjusting or removing the leg rests, and adjusting the foot/arm supports 50. This assists the person on the table as well as the therapist.

While embodiments of the invention have been described in the detailed description, the scope of the claims should not be limited by the preferred embodiments set forth in the examples but should be given the broadest interpretation consistent with the description as a whole.

What is claimed is:

1. A treatment table for a person to rest on having a first end with a removable head support section and with two foot/arm supports extending from first end of the treatment table wherein each foot/arm support is sized to support a forearm or foot.

2. The treatment table of claim 1, in which the foot/arm supports are adjustable outwardly and inwardly as well as upwardly and downwardly relative to the table.

3. The treatment table of claim 2, which additionally comprises a removable leg rest on each side of the table and wherein each leg rest is independently postionable along the length of the treatment table.

4. A treatment table comprising a leg rest on each side of the table that is independently postionable along the length of the treatment table.

5. The treatment table of claim 4, in which the leg rests are removable.

6. A treatment table with a head rest comprised of a first head rest section surrounded by a second head rest section wherein the first head rest section is removable and the second head rest section is on a spring loaded hinge bendable into an over 90 degrees downward angle from the original position.

7. The treatment table of claim 6 in which the first head rest section is replaceable with a tray.

8. The treatment table of claim 6 in which the first head rest section covers a tray which is accessible when the first head rest section is removed.

9. A treatment table comprising at least a first section and a second section wherein the first section and the second section are both independently adjustable to tilt towards each other.

10. The treatment table of claim 9 in which the second section is larger than the first section and the second section can tilt up to 90 degrees and the first section can tilt up to 20 degrees.

11. The treatment table of claim 10 which additionally comprises a head section.

12. The treatment table of claim 10 which additionally comprises removable leg rests which slide independently along each side of the table.

13. The treatment table of claim 12, which additionally comprises two foot/arm supports extending from the treatment table wherein each foot/arm support is sized to support a forearm or foot.

14. The treatment table of claim 13, in which the first section has concave contours on each side.

15. A treatment table comprising a first section at a first end and a second section at a second end, and two foot/arm supports extending from the first end of the treatment table, wherein the first section is adjustable to tilt upwards at the first end.

16. The treatment table of claim 15, in which the second section is additionally adjustable to tilt upwards at a second end.

17. The treatment table of claim 16, which additionally comprises a removable leg rest on each side of the table and wherein each leg rest is independently postionable along the length of the treatment table.

18. The treatment table of claim 17, in which the foot/arm supports are adjustable outwardly and inwardly as well as upwardly and downwardly relative to the table.

19. The treatment table of claim 18, which additionally comprises a removable head section.
